SEQUENCE LISTING (5 sequences)

SEQ ID NO: 1 - protein, 54 aa, Adenovirus type 37
KPMTFTINFNGLSETGDTPVSKYSMTFSWRWPNGSYIGHNFVTNSFTFSYIAQE

SEQ ID NO: 2 - protein, 63 aa, Adenovirus type 37
KPMTFTINFNGLSETGDCDCRGDCFCTPVSKYSMTFSWRWPNGSYIGHNFVTNSFTFSYIAQE

SEQ ID NO: 3 - protein, 61 aa, Adenovirus type 37
KPMTFTINFNGLSETGDYKDDDDKTPVSKYSMTFSWRWPNGSYIGHNFVTNSFTFSYIAQE

SEQ ID NO: 4 - protein, 54 aa, Adenovirus type 37
KPVTLTITLNGTQETGDTTPSAYSMSFSWDWSGHNYINEIFATSSYTFSYIAQE

SEQ ID NO: 5 - protein, 55 aa, Adenovirus type 37
KPMTFTINFNGLSETGDDTPVSKYSMTFSWRWPNGSYIGHNFVTNSFTFSYIAQE

(19) United States
(12) Patent Application Publication
(10) Pub. No.: US 2019/0201551 A1
(43) Pub. Date: Jul. 4, 2019

(54) PULMONARY TARGETED CAS9/CRISPR FOR IN VIVO EDITING OF DISEASE GENES

(71) Applicant: Washington University, Saint Louis, MO (US)

(72) Inventor: David CURIEL, Saint Louis, MO (US)

(73) Assignee: Washington University, Saint Louis, MO (US)

(21) Appl. No.: 16/303,790

(22) PCT Filed: May 23, 2017
(86) PCT No.: PCT/US17/34070

§ 371 Date: Nov. 21, 2018

Related U.S. Application Data
(60) Provisional application No. 62/340,501, filed on May 23, 2016.

(51) Int. Cl. A61K 48/00; A61P 7/04

(52) U.S. Cl. CPC A61K 48/0058; A61P 7/04; A61K 48/0008; A61K 48/0066

(57) ABSTRACT

An adenovirus vector comprising: a pulmonary targeting coding sequence, CRISPR components such as a Cas9 coding sequence, and a guideRNA coding sequence which can be used for gene therapy. The adenovirus can be a gorilla adenovirus, and can include a pulmonary cell targeting sequence such as an MBP targeting ligand coding sequence. The adenovirus can be targeted to pulmonary epithelium with a vascular specific promoter and integrin targeting peptides incorporated into a viral knob. The adenovirus can be used to introduce serum proteins via the pulmonary epithelium. Hemophilia can be treated by adenoviral introduction of factor VIII or factor IX.

### REFERENCE TO PRIOR APPLICATION

This application claims priority to and benefit of U.S. Provisional Application 62/340,501, filed on May 23, 2016. This application is hereby incorporated by reference in its entirety.

### STATEMENT OF GOVERNMENT SUPPORT

This invention was made with government support under GM103757, CA154697 and DE022957 awarded by the National Institutes of Health. The government has certain rights in the invention.

### INCORPORATION BY REFERENCE OF SEQUENCE LISTING

The Sequence Listing, which is a part of the present disclosure, includes a computer readable form and a written sequence listing comprising nucleotide and/or amino acid sequences. The sequence listing information recorded in computer readable form is identical to the written sequence listing. The subject matter of the Sequence Listing is incorporated herein by reference in its entirety

### INTRODUCTION

Gene therapy approaches for treatment of genetic diseases, such as hemophilia treatment through correction of Factor IX deficiency, face at least two significant challenges: stable, long-term expression, and reduction of hepatotoxicity. Long term correction of factor IX deficiency, via hepatic transduction with an adeno-associated vector (AAV), represents a watershed event in the history of the gene therapy field. In spite of this success, there presently exist barriers to the fullest implementation of hemophilia gene therapy. In the first regard, the limited packaging capacity of AAV for larger gene constructs practically confounds the use of this vector. In the second regard, vector-related toxicities have been most noteworthy for the context of in vivo transduction of the liver.

Adenovirus (Ad) has been employed principally for short term transient gene expression applications. However, methods have been developed to accomplish long term corrective gene expression via Ad. On this basis, integration capacities have been configured into Ad in an “in trans” manner allowing long term gene expression (transposons, homology sequences, etc.) (11-14).

Most recently, the advent of the CRISPR/Cas9 has provided a means to achieve effective gene editing in vivo via adenoviral vector-mediated transduction (15-18). A series of recent studies have validated that such targeted integration gene editing can be accomplished via vector-mediated in vivo delivery (19-22). It is noteworthy that all of these studies have been based upon liver transduction and none have yet sought to utilize other cellular sources in vivo. Additionally, Guan et al (EMBO Mol Med. 2016 Mar. 10. pii: e201506039. doi: 10.15252/emmm.201506039), used adenovirus to deliver Cas9 components and Factor IX corrections in mice, with liver targeting. They not only observed liver toxicity, but also failed to observe an increase in clotting ability.

U.S. Pat. Nos. 9,233,153, 9,629,906 and 9,617,560, all to GenVec, Inc. describe the use of Gorilla adenovirus vectors, but these patents do not describe the use of CRISPR/Cas9 or the correction of Factor IX in vivo.

### SUMMARY

The present inventor has developed vectors and methods for delivery of CRISPR/Cas9 components to cells in vivo. These vectors and methods can be used to deliver CRISPR/Cas9 components to targeted cells, and can also be used to effect transfer of therapeutic genes to a target cell. Delivery of CRISPR/Cas9 components to the relevant target cells can be used to modify genomes for therapeutic purposes. This delivery can be efficient, selective and coordinated.

The present inventor has combined targeted adenovirus with CRISPR/Cas9 to accomplish in vivo editing at specific organ sites. The process allows editing of specific gene loci for gene therapy and other applications. The present inventor, therefore, has designed a CRISPR/Cas9 delivery system that can be targeted to pulmonary cells, and can thereby bypass the liver and can also reduce toxicity. In some configurations, the vectors and methods can be used to transfer to a recipient a therapeutic gene, such as Factor IX, for treatment of hemophilia, while avoiding liver toxicity. In some configurations, the targeted cells can be pulmonary cells, and the targeted organ can be the lung.

The present teachings include integration-competent Ad which can provide gene expression longevity mandated for treatment of various genetic diseases, such as, for example hemophilia gene therapy.

In some embodiments, the present teachings can include, without limitation, an adenovirus vector comprising: a pulmonary targeting coding sequence, a Cas9 coding sequence, and a guideRNA coding sequence. In various configurations, the pulmonary targeting coding sequence can be a myeloid binding protein (MBP) targeting ligand coding sequence. In some configurations, the MBP targeting ligand coding sequence can be a fiber-fibritin-MBP targeting ligand coding sequence. In various configurations, the pulmonary targeting sequence can comprise a vascular specific promoter and an integrin targeting peptide incorporated into a viral knob of an adenovirus. In various configurations, the Cas9 coding sequence can be a codon-optimized. Cas9 coding sequence. In some configurations, the adenovirus vector can be a gorilla adenoviral vector. In some configurations, the guideRNA can comprise a Pol III promoter. In various configurations, the guideRNA can encode one or more sequences targeted to a ROSA26 locus.

In various embodiments, the present teachings can include a two-vector system comprising a first vector in accordance with the present teachings and a second vector comprising left and right homology arms that are homologous to the guideRNA. In some configurations, the second vector can further comprise a promoter. In some configurations, the promoter of the second vector can be a constitutive promoter. In some configurations, the constitutive promoter can be an EF1α promoter that can include an intron. In various configurations, the second vector can further comprise a multiple cloning site between the left and right homology arms configured such that when a sequence is inserted into the multiple cloning site, the second vector cannot be bound by Cas9. In various configurations, the second vector can further comprise a gene of interest. In some configurations, a gene of interest can be inserted into the multiple cloning site. In various configurations, the second vector can further comprise a Factor IX gene sequence. In various configurations, the pulmonary targeting coding sequence can be an MBP targeting ligand coding sequence. In various configurations, the guideRNA and the homology arms can comprise sequences targeted to the ROSA26 locus. In various configurations, the second vector can further comprise a Factor IX gene disrupting the sequence of the ROSA26 encoding homology arms. In various configurations, the pulmonary targeting coding sequence can be a fiber-fibritin-MBP targeting ligand coding sequence, the Cas9 coding sequence can be a codon optimized coding sequence, the adenovirus vector can be a gorilla adenovirus vector, the guideRNA can be under the control of the Pol III promoter. In some configurations, the guideRNA and homology arms can encode one or more sequences targeted to the ROSA26 locus and the second vector can further comprise a Factor IX gene which can disrupt the sequence of the ROSA26 encoding homology arms.

In various embodiments, the present teachings can include an adenovirus vector comprising a chimeric polypeptide comprising a de-knobbed adenovirus fiber, a T4 bacteriophage fibritin trimerizing foldon domain and a pulmonary targeting ligand. In some configurations, the de-knobbed adenovirus fiber can be a de-knobbed Ad5 fiber. In various configurations, the pulmonary targeting ligand can be a myeloid binding protein (MBP). In various configurations, the adenovirus can be a gorilla adenovirus. In various configurations, an adenovirus vector in accordance with the present teachings can further comprise a DNA sequence encoding a chimeric polypeptide comprising a de-knobbed Ad5 fiber, a T4 bacteriophage fibritin trimerizing foldon domain and a pulmonary targeting ligand. In various configurations, an adenovirus vector in accordance with the present teachings can further comprise a nucleic acid sequence encoding CRISPR components. In various configurations, an adenovirus vector in accordance with the present teachings can further comprise a DNA sequence encoding a Cas9. In various configurations, the DNA sequence encoding a Cas9 can be a mammalian codon-optimized sequence encoding a Cas9. In various configurations, an adenovirus vector in accordance with the present teachings can further comprise a promoter operably linked to the DNA sequence encoding a Cas9. In some configurations, the promoter operably linked to the DNA sequence encoding a Cas9 can be a CMV promoter. In various configurations, an adenovirus vector in accordance with the present teachings can further comprise a DNA sequence encoding a guideRNA (gRNA). In various configurations, an adenovirus vector in accordance with the present teachings can further comprise a promoter operably linked to the DNA sequence encoding a gRNA. In some configurations, the promoter operably linked to the DNA sequence encoding a gRNA can be a U6 promoter. In various configurations, an adenovirus vector in accordance with the present teachings can further comprise a DNA sequence targeted to an intronic region of a ROSA26 locus. In various configurations, an adenovirus vector in accordance with the present teachings can further comprise a DNA sequence targeted to the second intronic region of a ROSA26 locus.

In various embodiments, an adenovirus vector of the present teachings can comprise a plurality of integrin targeting peptides incorporated into an adenoviral fiber knob. In various configurations, an adenovirus vector in accordance with the present teachings can further comprise a T4 bacteriophage fibritin trimerizing foldon domain. In various configurations, an adenovirus vector in accordance with the present teachings can further comprise a vascular-specific promoter. In various configurations, the vascular-specific promoter can be a ROBO4 enhancer/promoter.

In various embodiments, an adenovirus vector of the present teachings can comprise: a chimeric AD5-T4 phage fibritin shaft, a trimerization domain displaying a pulmonary targeting coding sequence, and a ROBO4 enhancer/promoter operatively linked to a transgene of interest. In some configurations, the transgene of interest can be a CRISPR gene. In various configurations, the transgene of interest can be a Cas9 gene. In some configurations, the Cas9 gene can comprise a mammalian codon-optimized Cas9 coding sequence. In various configurations, the transgene of interest can be a guideRNA (gRNA). In various configurations, the guideRNA can comprise a Pol III promoter. In various configurations, the guideRNA can encode one or more sequences targeted to a ROSA26 locus. In various configurations, the transgene of interest can encode a hemophilia factor. In various configurations, the transgene of interest can encode hemophilia factor VIII or hemophilia factor IX. In various configurations, the adenovirus vector can be a gorilla adenoviral vector.

In various embodiments, the present teachings can include a two vector system comprising a first vector in accordance with the present teachings and a second vector comprising left and right homology arms that are homologous to a guideRNA. In some configurations, the second vector can further comprise a promoter. In various configurations, the promoter of the second vector can be a constitutive promoter. In various configurations, the constitutive promoter can be an EF1α promoter that includes an intron. In various configurations, the second vector can further comprise a multiple cloning site between the left and right homology arms configured such that when a sequence is inserted into the multiple cloning site, the second vector cannot be bound by Cas9. In various configurations, the second vector can further comprise a gene of interest. In various configurations, a gene of interest can be inserted into the multiple cloning site. In some configurations, the gene of interest can be a Factor IX gene. In various configurations, the pulmonary targeting coding sequence can be an MBP targeting ligand coding sequence. In various configurations, the guideRNA and the homology arms can comprise sequences targeted to the ROSA26 locus. In various configurations, the second vector of a two vector system in accordance with the present teachings can further comprise a Factor IX gene which disrupts the sequence of the ROSA26 encoding homology arms. In some configurations, the pulmonary targeting coding sequence can be a fiber-fibritin-MBP targeting ligand coding sequence, the adenovirus vector can be a gorilla adenoviral vector, the guideRNA can be under the control of the Pol III promoter, the guideRNA and homology arms can encode one or more sequences targeted to a ROSA26 locus, and the second vector can further comprise a Factor IX gene disrupting the sequence of the ROSA26 encoding homology arms.

In various embodiments, a method of gene therapy can comprise administering to a subject an adenovirus comprising a two vector system in accordance with the present teachings. In some configurations, the adenovirus can be a gorilla adenovirus.

In various embodiments, the present teachings can include an adenovirus vector comprising a pulmonary targeting ligand, a vascular-specific promoter and integrin targeting peptides incorporated into a viral knob.

### BRIEF DESCRIPTION OF THE DRAWINGS

FIG. 1 illustrates adenoviral retargeting via fiber replacement of the native Ad5 fiber protein. Normally Ad binds to the Coxsackie-adenovirus-receptor (CAR) via the knob domain of the fiber protein.

FIG. 2A illustrates MBP peptide targeting of adenovirus to lung.

FIG. 2B illustrates targeted gene transfer localized to alveolar capillaries.

FIG. 3 illustrates serum A1AT levels achieved via targeted and untargeted adenoviral vectors.

FIG. 4 illustrates four GAd46 vectors containing the MBP targeting peptide.

FIG. 5 illustrates a ribbon diagram of the Gorilla Ad Fiber Domain.

FIG. 6 illustrates incorporation of RGD-4C or flag peptide within the III-loop of the gorilla Ad fiber knob domain.

FIG. 7 illustrates a polyacrylamide gel electrophoresis analysis of modified fiber (RGD or Flag peptide) incorporation into gorilla Ad capsid.

FIG. 8 illustrates a Western blot against anti-flag mAb, M2.

FIG. 9 illustrates a Western blot against anti-fiber tail mAb, 4D2.

FIG. 10 illustrates analysis of RGD-4C peptide incorporation—Ad (acne transfer efficiency.

FIG. 11 illustrates analysis of RGD-4C peptide incorporation—Gorilla Ad gene transfer efficiency.

FIG. 12 illustrates analysis of RGD-4C peptide incorporation—Gorilla Ad gene transfer efficiency.

FIG. 13 illustrates analysis of RGD-4C peptide incorporation—Gorilla Ad gene transfer efficiency.

FIG. 14 illustrates that Ad.MBP preferentially targets lung in vivo by charting biodistribution of Ad.MBP.Luc Expression in C57BL/6J mice.

FIG. 15 illustrates preferential targeting of Ad.MBP to lung in vivo.

FIG. 16 illustrates non-Gorilla adenovirus vectors Ad.CMVCas9, Ad.U6gRNA, Ad.CMVCas-9-UgRNA, Ad5.EF1eGFP (Rosa26 donor), Ad5-αmFIX (Rosa26 donor) and Ad5.EF1αhA1AT (Rosa 26 donor).

FIG. 17 illustrates that adenoviral vector-mediated delivery of CRISPR/Cas9 results in non-homologous end joining in-vitro through INDEL formation at ROSA26 target locus using Ad5.CMV-Cas9-gRNA transduced into BNL-1NG cells.

FIG. 18 illustrates adenoviral vector-mediated delivery of CRISPR/Cas9 results in non-homologous end joining in vivo through INDEL formation in an Ad5Cas9gRNA injected CB57lJ mouse.

FIG. 19 illustrates a FIX Western blot of supernatants of A549 cells with ADEF1amF9 (Rosa26 donor) at 1000 and 5000 MOI, at 72 hours post injection.

FIG. 20 illustrates A549 cells infected with Ad5.EF1aGFP donor (10000 vp/cell).

FIG. 21 illustrates amount of GFP expressing cells 50 days after infection for various cell lines.

FIG. 22 illustrates targeted integration of hA1AT into ROSA26 locus permits extended gene expression via co-delivery CRISPR/Cas9 and Donor Vector in vivo via ELISA.

FIG. 23 illustrates mouse livers show extended GFP expression from both integrated genomic and episomal DNA at week 1 (left column) and week 6 (right column).

### DETAILED DESCRIPTION

Whereas viral vectors have been employed for in vivo delivery of CRISPR/Cas9, these applications have been limited by the native tropism of the viral vector used. The present teachings include modification of adenovirus to alter targeting to selected host cells, combined with CRISPR/Cas9 delivery for editing of targeted cells.

The packaging capacity of Ad allows vector incorporation of all CRISPR/Cas9 elements ensuring coordinated functionality. The ability to modify Ad tropism can facilitate in vivo targeting. Targeted Ad thus embodies a set of attributes which can be useful for targeted in vivo editing.

The present inventor has developed an approach based upon gene delivery to the blood vessels of the lung. This strategy provides an alternative to liver sourcing of corrective hemophilia factors and can allow the application of gene therapy for diseases such as, for example, hemophilia, while avoiding liver toxicity. In some configurations, in vivo transduction of the pulmonary endothelium via targeted Ad can demonstrate that pulmonary endothelium can serve as an effective platform to reconstitute deficient serum proteins. In some configurations, an Adenovirus can be modified to incorporate a range of gene constructs relevant to gene therapy for the full spectrum of hemophilia disorders.

In various embodiments, Ad technology can be fully commensurate with the CRISPR/Cas9 system, and can allow in vivo genetic modifications for stable gene expression.

Various embodiments include utilizing the CRISPR/Cas9 system in conjunction with pulmonary endothelial-targeted Ad, to achieve stable incorporation of corrective genes within the pulmonary endothelium, such as, for example, genes that can correct hemophilia. In some configurations, the strategy can provide for stable genetic correction of hemophilia in a manner that can circumvent potential vector-associated toxicities for both factor IX and factor XIII hemophilia gene therapy. This approach thereby facilitates the application of corrective gene therapy for hemophilia.

In various configurations, an adenovirus vector of the present teachings can be used to effect stable expression from a pulmonary vascular source of introduced genes such as deficient hemophilia factors, while circumventing vector-mediated hepatotoxicities. In various configurations, an adenovirus vector of the present teachings can be used for gene therapy for deficiency disorders for serum proteins such as, without limitation, factor VIII or factor IX.

In some embodiments, an adenovirus of the present teachings can be an adenovirus of a non-human primate such as a gorilla (Gad). In some configurations, adenovirus from a non-human primate can be used to avoid reactions of a human subject against human serotype 5-based Ad, including reactions due to preformed antibodies against human serotype 5-based Ad.

### Methods

The methods and compositions described herein utilize laboratory techniques well known to skilled artisans, and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; Nagy, A., Manipulating the Mouse Embryo: A Laboratory Manual (Third Edition), Cold Spring Harbor, N.Y., 2003 and Harlow, E., Using Antibodies: A Laboratory Manual, Cold Spring Harbor Laboratory Press, Cold Spring Harbor, N.Y., 1999. Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003. As used in the present description and the appended claims, the singular forms “a”, “an” and “the” are intended to include the plural forms as well, unless the context indicates otherwise.

### EXAMPLES

The present teachings include descriptions provided in the examples that are not intended to limit the scope of any aspect or claim. Unless specifically presented in the past tense, an example can be a prophetic or an actual example. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Example 1

This example illustrates tropism modification of adenovirus via genetic capsid modification.

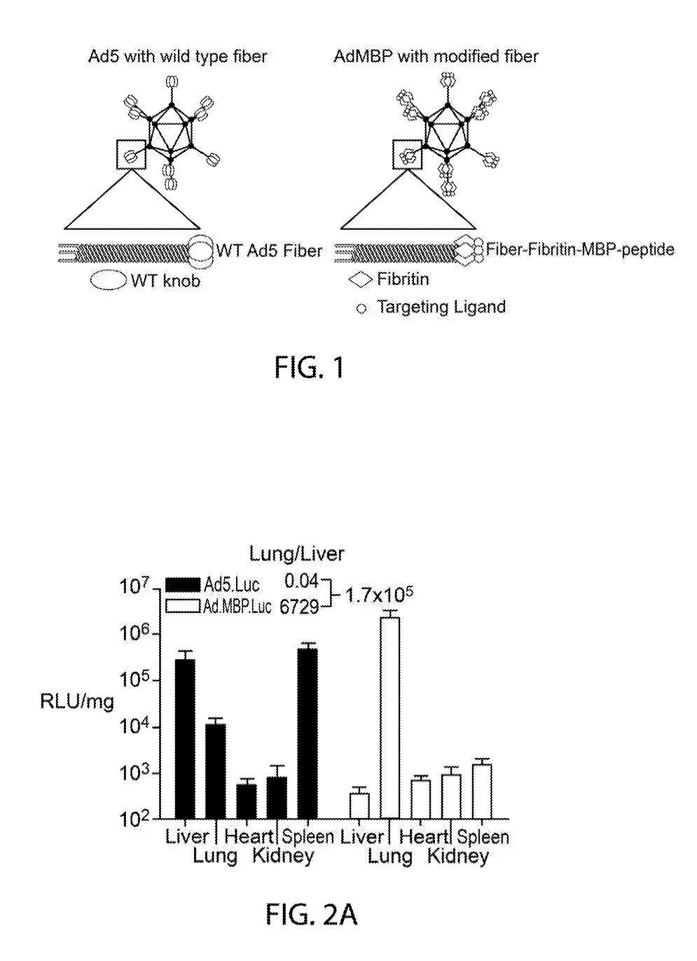

The present inventor altered tropism of Ad via genetic incorporation of binding ligands within specific domains of the adenoviral fiber protein (1). The present inventor has replaced the native fiber protein with a chimera of fiber plus the T4 Pol protein fibritin (2, 3). To accomplish Ad vector retargeting, the fiber protein is replaced by a chimera consisting of the tail domain of the native fiber fused to the T4 Pol protein fibritin, and an MBP peptide binding to myeloid cells. Utilizing this method the inventor incorporated into the Ad capsid the peptide ligand myeloid binding protein “MBP” (FIG. 1). Studies have validated the specificity of the AdMBP to accomplish efficient and selective gene delivery to cells of myeloid lineage (4). We have used this system to test our hypothesis with respect to pulmonary vascular transduction via a “hitchhiking” mechanism (5). This system is an excellent mechanism for Cas/Crisper Ad vector virus delivery.

### Example 2

This example illustrates targeted gene transfer to pulmonary vascular endothelium via a tropism-modified adenoviral vector.

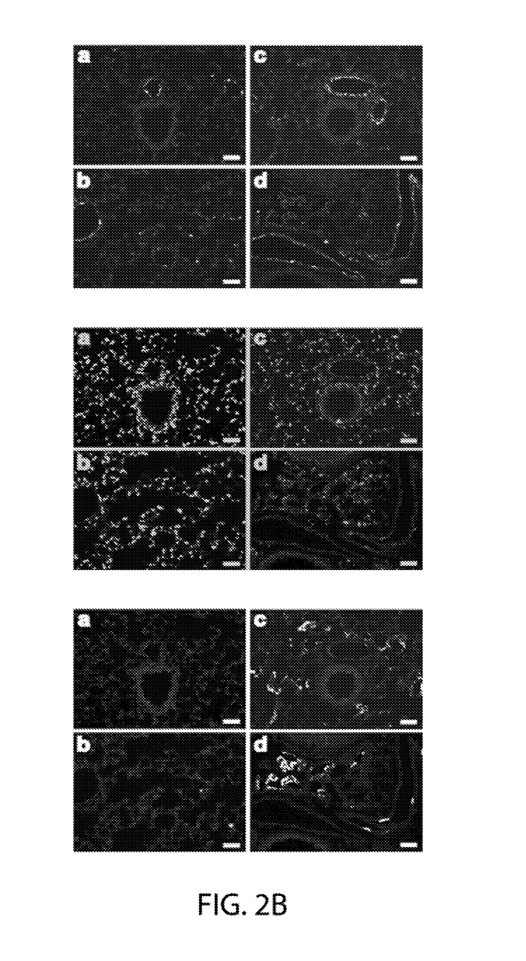

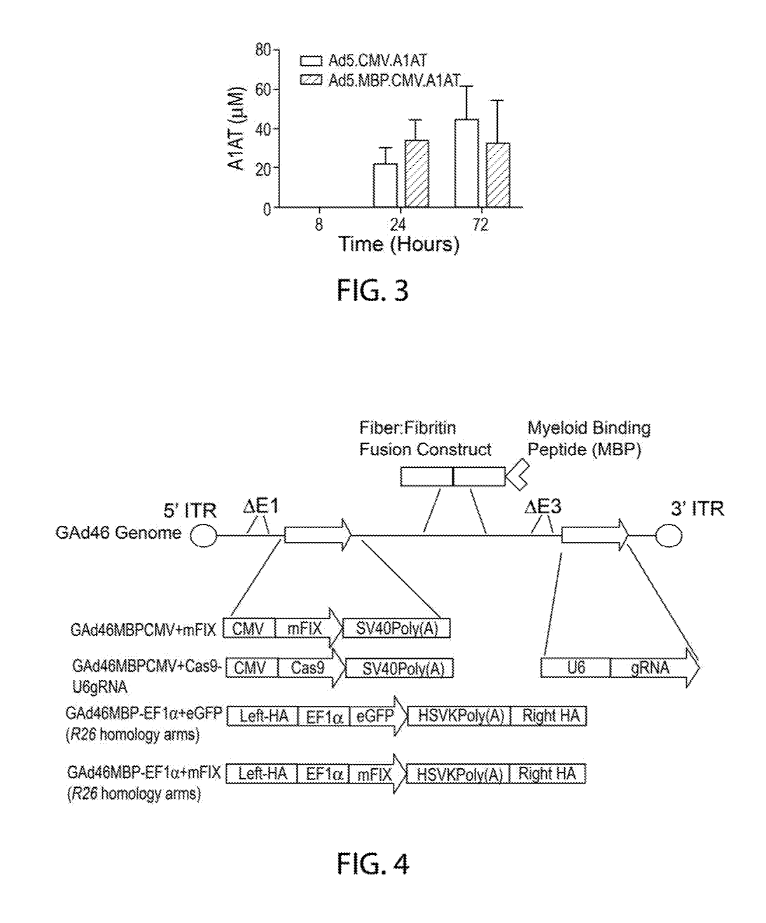

In these experiments, mice were given via tail vein conventional untargeted huAd5 vector encoding reporter (Ad5.Luc) or tropism modified Ad with capsid incorporated MBP (Ad.MBP.Luc). As can be seen in FIG. 2A and FIG. 2B, Ad.MBP.Luc exhibited a statistically significant targeting effect with enhancement of gene delivery to the pulmonary endothelium and reduction of gene transfer to the liver, in comparison to Ad5.Luc. Ad.MBP.Luc and Ad.Luc were injected in C57BL6 mice via tail vein. For FIG. 2A tissues were collected 24 hours later and assayed for luciferase expression in lysate from various organs normalized to mg of total protein. For FIG. 2B, mice were injected (i.v.) with 2.5×10¹⁰ viral particles (VP) of MBP-targeted Ad encoding the green protein reporter, Ad.MBP.GFP, 24 hours prior to sacrifice. Panels have been split into color channels in order to render the figure in black and white. Panels show staining for the GFP transgene (FIG. 2B, bottom panel), von Willebrand factor (FIG. 2B, top panel), and nuclei (DAPI, FIG. 3B, middle panel) in (a) uninjected controls, and mice injected with (b) control Ad5.GFP, or (c, d) Ad.MBP.GFP. Scale bar, Images are representative of n=2-3 mice per group. Of note, the increase in the targeting ratio was more than five orders of magnitude compared to non-targeted adenovirus. The addition of MBP specifically targets Ad to lung tissue.

### Example 3

Others have highlighted the potential of extrahepatic sources of coagulation factors for gene therapy (6). This example illustrates that pulmonary endothelium can serve as a cellular source of a secretory protein to augment serum levels.

In these experiments, the present inventor utilized alpha1-antitrypsin (A1AT) which has biosynthesis and biodistribution analogies to serum coagulation factors. For these studies, mice were administered intravenously untargeted, or MBP-targeted Ad encoding human A1AT. At intervals post-challenge, serum A1AT was analyzed (7). As can be seen in FIG. 3, the targeted Ad could augment serum A1AT in a manner comparable to the control Ad. C57BL/6J mice were injected via tail vein with either 10¹⁰ viral particles of Ad5.CMV.A1AT or 10¹⁰ viral particles of Ad5.MBP.CMV.A1AT. The mice were then sacrificed 8, 24 or 72 hours later, and their serum was collected to determine A1AT. n=5 mice in each group at each time point. No A1AT was detectable in the serum at 8 hours. Both groups of mice had detectable serum concentrations at 24 and 72 hours, but there was no significant difference in serum A1AT concentration. These studies illustrate that pulmonary vasculature “sourcing” of a secretory protein can allow significant augmentation of serum levels.

The inventor has thus established herein: 1) modification of the tropism of adenoviral vectors via fiber replacement with the capsid incorporation of targeting peptides; 2) that Ad modified to accomplish myeloid cell binding achieves effective targeting to the pulmonary endothelium and untargeting of the liver in vivo in murine models; and 3) that a pulmonary endothelial-sourced serum protein can directly augment serum levels.

In some embodiments, the present teachings include gorilla Ad with similar tropism modifications. Such vectors can circumvent preformed immunity to huAd5 and can allow treatment of patients irrespective of anti-Ad5 immunity. Pulmonary endothelium can thus be used as a cellular source for a serum protein such as factor VIII/factor IX. The present teachings include integration-competent Ad which can provide gene expression longevity mandated for treatment of various genetic diseases, such as, for example hemophilia gene therapy.

### Example 4

This example illustrates the construction of a gorilla adenoviral vector (GAd) targeted to the pulmonary endothelium, and demonstration of augmentation of Factor IX (FIX) levels in FIX hemophilia deficient mice.

In these experiments, GAd targeted to the pulmonary endothelium are constructed. The capacity of such targeted vectors to accomplish corrective levels of FIX in FIX hemophilia mice can be demonstrated.

Construction of tropism-modified gorilla adenoviral vector incorporating myeloid binding peptide (MBP). In some configurations, a two-plasmid rescue method can be employed to construct a fiber-modified GAd incorporating a myeloid cell targeted heptapeptide into a de-knobbed huAd5 fiber chimera with the trimerizing foldon domain of bacteriophage T4 fibritin (see FIG. 1). The recombinant GAd genome encoding chimeric fiber fused with MBP ligand (pGAd46FF-MBP) can be derived by homologous recombination in E. coli strain BJ5183 between a plasmid carrying the GAd46 genome lacking the fiber gene and a DNA fragment containing a fiber-fibritin-MBP (FF-MBP) gene as previously described (2,4). E3 genes and the downstream fiber gene sequences can be deleted in the GAd46 genomic plasmid DNA and can be used for recombination with a shuttle plasmid carrying the chimeric fiber-fibritin-MBP gene flanked by surrounding GAd46 genome sequences. The FIX gene cDNA can be cloned from the plasmid pGEMmFIX (Sino Biological Inc., Beijing, China). This ORF can be ligated into a shuttle plasmid carrying the CMV promoter connected to the SV40 pA via MCS and can be flanked by surrounding GAd46 genome sequences to generate pShuttleCMV.mFIX. The recombinant GAd genome encoding CMV-FIX-pA cassette can be derived by homologous recombination in E. coli strain BJ5183 between the plasmid carrying the GAd46FF-MBP genome but lacking the E1 gene, and the pShuttleCMV.mFIX employing methods previously described (8) (See FIG. 4). Viral particles (vp) can be quantified by measuring absorbance of the dissociated virus at A260 nm using a conversion factor of 1.1×10¹² vp per absorbance unit (9). For validation of genetic incorporation of binding peptides into fiber-fibritin purified GAd46FF-MBP viral particles can be analyzed by PCR and Western blot. These analyses can show that FIX is expressed in the lung but not the liver.

### Example 5

This example illustrates validation of myeloid binding specificity of tropism by modified GAd. For characterization of GAd binding specificity, BMCs can be isolated and in vitro GAd binding and transduction can be evaluated as previously described (4, 5). Cells can be infected with GAd vectors at various multiplicities of infection and cellular-bound GAds can be detected as previously described (4). Additionally, the concentrations of FIX in the media can be determined with the Factor IX Murine ELISA Kit Quantification Kit (Cloud-Clone Corp., Atlanta, Ga.). For blocking experiments, 1×10⁶ BMCs from FIX KO mice can be pre-incubated with 25 μg of MBP peptide for 30 min at 4° C. before addition of virus.

### Example 6

This example illustrates an analysis of biodistribution of tropism-modified GAd in murine models. In these experiments, FIX KO mice can be injected via tail vein with PBS alone, 1×10¹⁰ vp of GAd46FF-MBP-mFIX, GAd46 expressing wild type fiber or GAd46FF vector expressing fiber-fibritin alone. The mice can be sacrificed 2, 24 or 72 hours later, and lungs, livers and spleens can be collected. The tissues can be homogenized in PBS, and genomic DNA can be extracted with the DNeasy Blood and Tissue Kits (Qiagen). The relative amount of viral. DNA copies per ng of tissue genomic DNA can be determined by quantitative PCR (q-PCR). The mouse β-actin gene can be used as an internal standard for template loading of q-PCR. The q-PCR can show that FIX is expressed in the lungs, but not the spleen or liver.

### Example 7

Analysis of gene transfer targeting via tropism modified GAd in marine models. FIX KO mice can be injected via tail vein, as noted above. The mice can be sacrificed 2, 24 or 72 hours later, and blood can be collected and spun down allowing collection of the serum. The concentrations of mFIX in the serum can be determined with a Factor IX Murine ELISA Kit Quantification Kit.

The inventor's strategy to source FIX from the pulmonary endothelium can be based on in vivo gene delivery to this target cell.

### Example 8

The inventor has also developed alternative targeting methods compatible with GAds. The inventor has developed a “combination targeting” method (10) based upon a vascular-specific promoter in combination with integrin targeting peptides incorporated into the adenoviral fiber knob. This approach can achieve a high ratio of lung-to-liver targeting with transgene expression induced in vivo at the pulmonary endothelium.

### Example 9

This example illustrates the functional configuration of the CRISPR/Cas9 into pulmonary targeted GAd and demonstrate in vivo gene editing of target cells of the pulmonary endothelium.

The levels of targeted in vivo gene transfer can allow corrective gene editing that can be used for hemophilia gene therapy. Additionally, Ad's transient expression of CRISPR components can provide an additional level of safety whereby nuclease activity is restricted to a short temporal span thereby reducing potential genotoxicities. The present teachings include Ad-incorporated CRISPR/Cas9 for in vivo gene editing within pulmonary endothelial target cells. Feasibility studies can be demonstrated utilizing a GFP reporter. Studies herein can thus validate in vivo gene editing via genomic analysis as well as stable genetic analysis via monitoring of the incorporated reporter gene.

### Example 10

A two-vector strategy for GAd-based targeted gene editing of ROSA26 safe harbor locus. A two-vector strategy can be used in which one GAd vector encodes all necessary components of the Type II CRISPR/Cas9 gene editing system, targeted to the in urine ROSA26 ‘safe harbor’ locus, and a second vector encodes a constitutively expressed green fluorescent reporter gene with appropriate homology arms for integration. Non-limiting examples of these vectors are illustrated in FIG. 4, including a transient vector encoding mFIX, a vector encoding CRISPR components and a GFP reporter donor vector with Rosa26 homology arms for targeted genomic integration, and a Rosa26 donor vector encoding mFIX. This approach utilizes three plasmids (provided by Genome Engineering Core (GEC) at Washington University in St. Louis). The first plasmid encodes mammalian codon-optimized Cas9 and a nuclear leading sequence. This plasmid is used to clone Cas9 gene into the MCS of our shuttle plasmid (see example 3), resulting in pShuttle-CMV-Cas9. The pShuttle-CMV-Cas9 is recombined into the E1 region of our E1,E3-deleted GAd plasmid DNA upon co-transformation in Escherichia coli strain BJ518359. The plasmid pGAd46MBP-CMVCas9 is digested to release the viral genome followed by transfection into HEK293 cells expressing E1, allowing replication of GAd. Next, the replication defective virus is amplified, purified via two ultracentrifugations of CsCl gradients, and sequence validated using our standard techniques.

The second plasmid encodes an efficient guideRNA (gRNA), under the control of the Pol III promoter U6, targeting the second intronic region of the ROSA26 locus and can be used for the generation of transgenic mice. To obtain a vector expressing both CRISPR components, the U6gRNA expression cassette can be ligated into pShuttle-CMV-Cas9 to generate pShuttle-CMV-Cas9-U6-gRNA. The pShuttle-CMV-Cas9-U6-gRNA can be homologously recombined into the E1 region of our E1,E3-deleted GAd plasmid. DNA containing the MBP targeting ligand, as above. The resulting plasmid pGAd46MBP-Cas9gRNA can be transfected into 293 cells for rescue, amplified, purified, and sequence verified as described above (FIG. 4). The third plasmid encodes donor homology arm (HA) sequences composing the left and right sequences (app. 800 bp ea.) surrounding the ROSA26gRNA target site, modified by a MCS between the HAs. This MCS destroys the gRNA target site on the donor plasmid protecting it from cleavage by Cas9. This donor plasmid can also be used as a ROSA26 donor. The reporter eGFP can be inserted into pBApo (Clontech Laboratories, Mountain View, Calif.) under the control of the constitutive mammalian EF1α promoter which includes an intron. This combination of promoter, transgene and polyA signal cassette can be ligated into the ROSA26 donor plasmid and cloned into a GAd46 pShuttle. This shuttle vector can be recombined into a E1 deleted region to yield GAd46MBPEF1αGFP (Rosa26 Donor), rescued, amplified, purified, and sequence verified as above (see FIG. 4). These vectors can be used to transform genes such as FIX into mammalian organisms.

### Example 11

Functional Validation of Targeted Gene Editing by Genome Analysis.

To validate expression and function of these vectors, titers can be determined as described in Example 3. Gene products can be verified in vitro following infection with individual vectors at a MOI of 100 vp/cell. The Cas9 can be detected via western blot, gRNA production can be verified with qRT-PCR with a probe specific to the RNA scaffold, and eGFP can be detected via florescence microscopy. Double stranded cleavage at the ROSA26 target sequence and integration can be validated in vitro using a murine BNL-1NG cell line, GAdCas9gRNA will be infected at MOIs of 1, 5, and 10 thousand viral particles/cell. 48 hrs post-infection (hpi), cells can be harvested, whole genomic DNA extracted and used for generation of a PCR library for deep sequencing analysis to detect mutations at the ROSA26 target site. The PCR primers consist of 5′ and 3′ oligos flanking the gRNA target sequence. To validate integration at the ROSA26 locus, BNL-1NG cells can be infected with Gad46Cas9gRNA and GAd46EF1αeGFP (ROSA26 Donor) at a previously determined optimal cleavage MOI. Cell cultures can be passaged and monitored for stable eGFP expression over a period longer (4+ weeks) than is expected for transient unintegrated gene expression using fluorescent microscopy. Total integrated GFP expression in cells after multiple passages can be quantified using ImageJ software for relative florescent intensities compared to control infected and uninfected cell cultures. Following this period of extended culture, GFP+ cells can be sorted using flow cytometry and additional evidence of integration can be acquired using PCR amplification of the ROSA26 genomic region and southern blotting with the target site-specific probe to analyze migration differences due to insertions. These experiments can show that vectors of the present teachings are specifically targeted to lung tissue.

### Example 12

Functional Validation of Targeted Gene Editing with Reporter Gene Analysis.

Examination of in vivo efficacy of a fully targeted integration system can be performed. In these experiments, extended gene expression levels and integration rates following co-infection of GAd46Cas9gRNA and GAd46EF1αGFP in vivo can be analyzed using C57BL/J mice. Use of the GFP donor can allow optimization of an integration scheme without the added difficulty associated with handling hemophilia mice, which more readily die after injury (e.g. injection or blood draw) than wild type mice. For this analysis, wild-type C57BL/6 mice grown to 8-10 weeks old can be co-injected with 100 μL of PBS without virus or PBS containing equal amounts (10¹⁰, 5×10¹⁰, or 10¹¹ total viral particles) of the CRISPR encoding vector and the donor vector Gad46MBP-EF1^{α}eGFP (w/ R26 homology arms) through tail veins. Initially, a ratio of 1:1 of each vector can be utilized but can subsequently be altered to optimize in vivo genomic integration. There can be 3 dose groups each containing 8 C57BL/6 mice and 4 uninfected mice as a negative control. Additionally, 3 wild-type mice can be treated with GAd46MBPEF1^{α}GFP donor only (no nuclease) to serve as an experimental control for homology-directed repair (HDR) independent integration, per dose group. These mice can be sacrificed 4, 8, and 16 wks post infection and whole genomic DNA can be extracted from lung cells for analysis of integration rates using LAM-PCR for an unbiased amplification of the target genomic locus (ROSA26) of both integrated and non-integrated alleles, as described elsewhere (23), These PCR products can be used for qPCR quantification of the integration rates at the ROSA26 locus using separate qPCR reactions of non-integrated and integrated specific probes; B-actin can serve as an internal control to normalize the abundance of integrated vs non-integrated alleles. Additionally, liver tissue sections can be generated for immunofluorescent staining of eGFP positive cells to visualize expression resulting from integration. Injections and data analysis can be performed in a double blinded manner to ensure an unbiased experimentation and analysis of this strategy. Extraction RNA from lung tissue of treated and non-treated mice at the above time points can be performed to quantify GFP gene expression using GFP specific RT-PCR probes, with B-actin probes to normalize GFP mRNA quantity.

Additionally, injection of new born mice can increase homology-directed repair. Administration of a small molecule drug (Scr7) can inhibit non-homologous end joining (NHEJ) and increase HDR rate.

These experiments can demonstrate the ability of vectors of the present teachings to incorporate into the ROSA26 locus.

### Example 13

This example illustrates the use of a composite GAd vector system, which includes pulmonary vascular targeting and CRISPR/Cas9. This system can be used for gene therapy correction of hemophilia in a murine model, and can achieve stable, long term correction of factor IX deficient mice. Pulmonary vascular endothelium can serve as a cellular source for FIX to achieve correction levels of serum reconstitution. In vivo editing of pulmonary endothelial cells can be achieved via vector-mediated delivery of components of the CRISPR/Cas9 system. Based on these key feasibilities, gene therapy correction in a mammalian model of a genetic disease can be achieved. In the aggregate, vectors and methods of the present teachings can be used for treatment of genetic diseases such as hemophilia in humans.

### Example 14

Generation of a ROSA26 targeted donor vector encoding murine FIX. A mFIX donor vector targeted for gene insertion at the murine ROSA26 locus can be produced in a similar manner to the eGFP donor vector in Example 2. Briefly, mFIX cDNA (Sino Biological Inc) can be inserted into pBApo (Clontech Laboratories) under the control of the constitutive mammalian EF1α promoter which includes an intron. This promoter, transgene, and polyA signal cassette can be ligated into the ROSA26 donor plasmid and cloned into a GAd46 pShuttle. This shuttle vector is recombined into E1 deleted region to yield GAd46MBPEF1αmFIX (Rosa26 Donor), rescued, amplified, purified, and sequence verified as above (see FIG. 4). Transgene expression of this donor vector can be confirmed in human A549 cells following transduction at 1000 MOI, followed by Western Blot detection of the secreted mFIX in the culture supernatant. These experiments can illustrate that FIX can be transformed into the ROSA26 locus.

### Example 15

Functional Validation of Targeted In Vivo Gene Editing by Genome Analysis. FIX KO mice grown to 8-10 weeks old are co-injected with 100 μL of PBS without virus or PBS containing equal amounts (10¹⁰, 5×10¹⁰, or 10¹¹ total viral particles) of the CRISPR encoding vector and the donor vector Gad46MBP-EF1αmFIX (w/ R26 homology arms) through tail veins. 3 dose groups can each contain 8 C57BL/6 mice and 4 uninfected mice as a negative control. Additionally, 3 wild-type mice can be treated with GAd46MBPEF1αmFIX donor only (no nuclease) as an experimental control for HDR independent integration, per dose group. These mice can be sacrificed 4, 8, and 16 wks post infection and whole genomic DNA can be extracted from lung cells to analyze integration rates using LAM-PCR for an unbiased amplification of the target genomic locus (ROSA26) of both integrated and non integrated alleles, as described above and elsewhere (23). Lung tissue can be harvested for Whole protein extract for Western. Blot detection of mFIX production as well as generation of liver tissue sections for immunofluorescent staining of mFIX positive cells to visualize expression resulting from integration. Further evidence can be obtained by extracting RNA from lung tissue of treated and non-treated mice at the above time points to quantify mFIX gene expression using mFIX-specific RT-PCR probes, with B-actin probes to normalize GFP mRNA quantity. These experiments can illustrate that FIX can be transformed into the ROSA26 locus.

### Example 16

Validation of Phenotype Rescue in Targeted Gene Editing in hemophilic mice. Factor IX KO mice (8-10 weeks old) can be co-injected with 100 μL of PBS without virus or PBS containing equal amounts (10⁹, 10¹⁰, or 10¹¹ total viral particles) of the CRISPR encoding vector and the donor vector Gad46MBP-EF1αmFIX (w/ R26 homology arms) through tail veins. Scheduled blood samples can be collected from the retro-orbital plexus and the plasma can be stored at −80° C. for future ELISAs of mFIX levels and activated partial thromboplastin time (APTT) assays to measure FIX coagulation activity. Blood collection can occur at 24 hrs, 48 hrs, 1 wk, and every two weeks thereafter for up to 60 weeks (or until mFIX is no longer detectable) post-administration. Plasma can also be collected in parallel from untreated mice and wild-type C57BL/6 mice of the same age to serve as negative and positive controls, respectively. Additional experimental evidence can be obtained using an AAV vector encoding murine FIX. 3 dose groups each contain 15 KO mice and 4 mice (C57BL/6) as a positive control. 3 FIX KO mice can be treated with GAd46MBPEF1αmFIX donor only (no nuclease) to serve as an experimental control for HDR independent integration, per dose group. The total for the experiment can be therefore 54 FIX KU mice and 12 C57Bl/6 mice. 3 randomly selected mice of each hemophiliac background in each dose group can be subjected to a tail bleed assay at 8 weeks post administration for further validation of increased coagulation ability with non-treated and wild type mice used as controls. These experiments can show that vectors of the present teachings can be used to rescue the hemophilia phenotype.

### Example 17

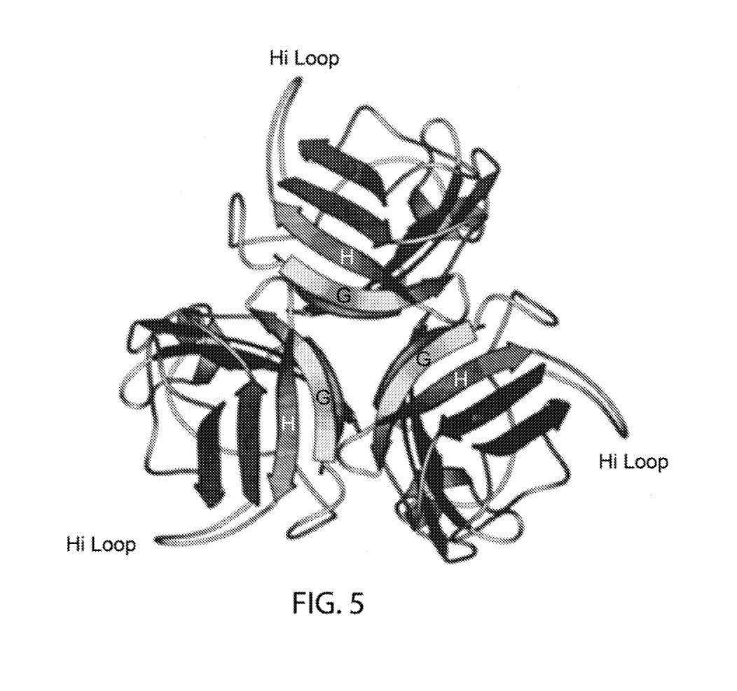

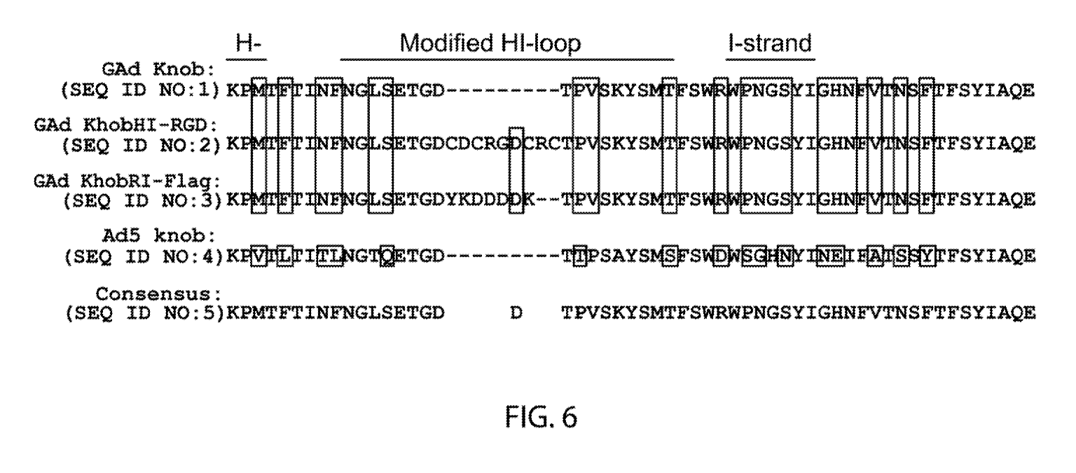

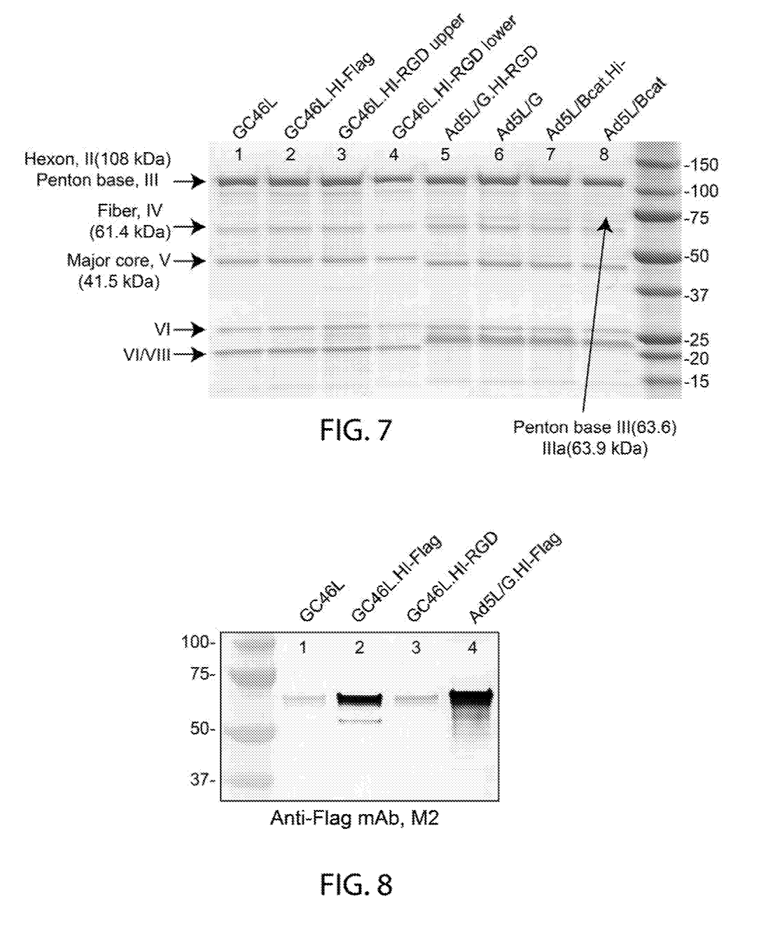

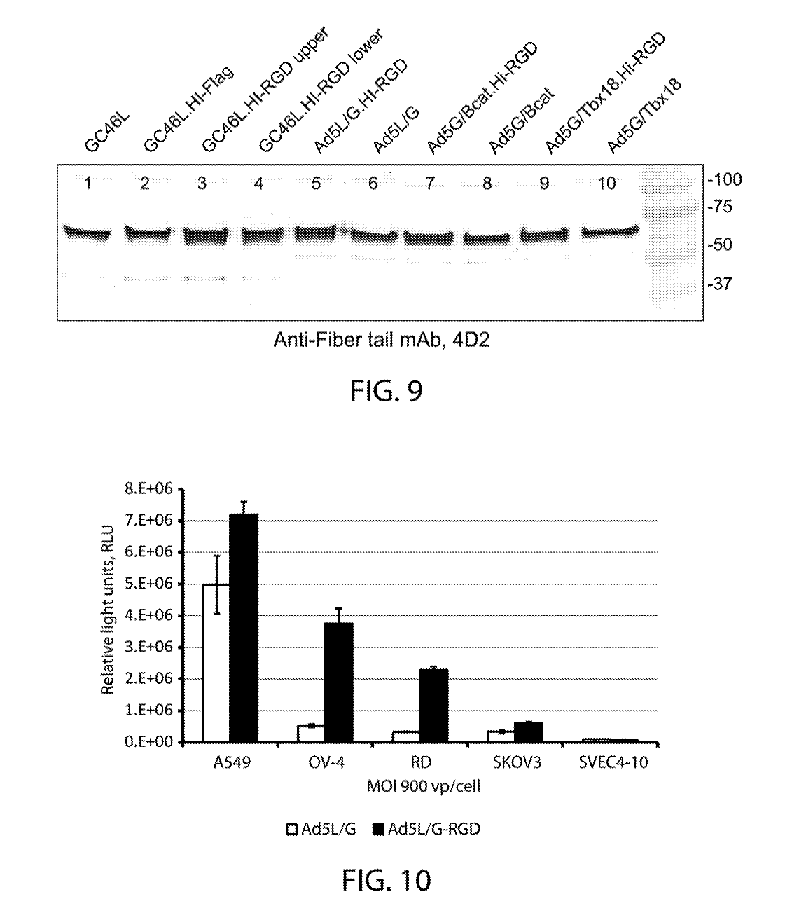

This example illustrates Gorilla Ad Fiber Knob domain gene transfer efficiency. FIG. 5 illustrates the 3-dimensional structure of the Gorilla Ad fiber knob, including the location of the HI loops. FIG. 6 illustrates the incorporation of RGD-4C (GAd KnobHI-RGD) and FLAG (GAd Knob HI-Flag) tags into the HI loop of the gorilla adenovirus knob. FIG. 7 illustrates total protein staining of gel purified. Gorilla Ad. FIG. 8 illustrates an anti-flag Western blot of flag tagged Gorilla adenovirus knobs derived from gel purified Gorilla Ad. FIG. 9 illustrates a Western blot detected with anti-fiber tail antibody, as before.

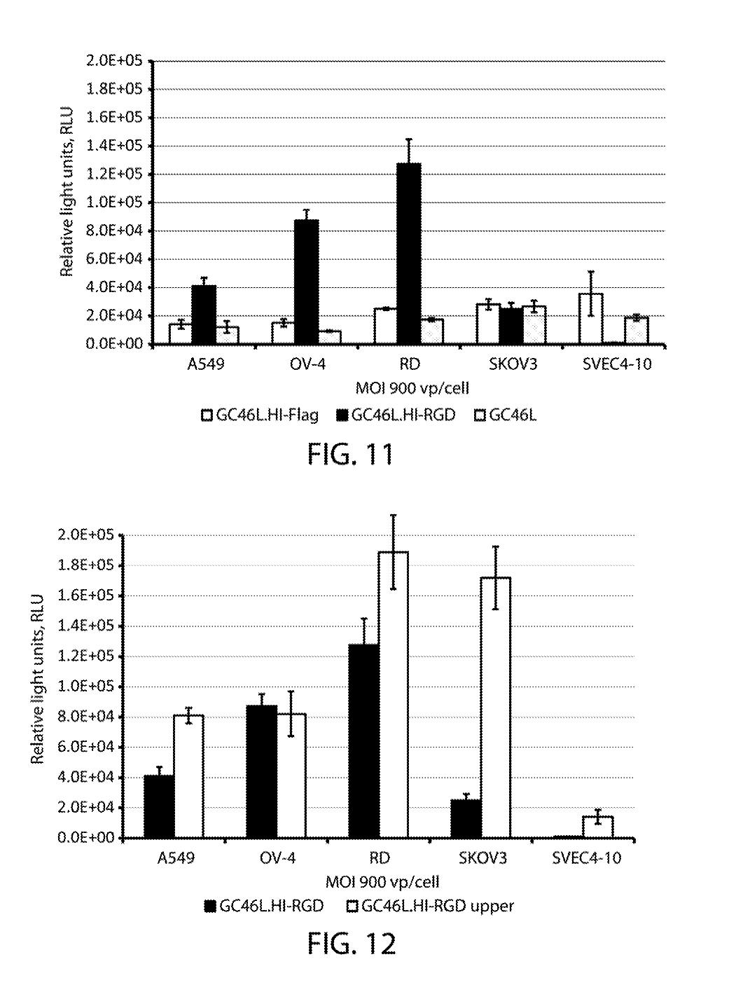

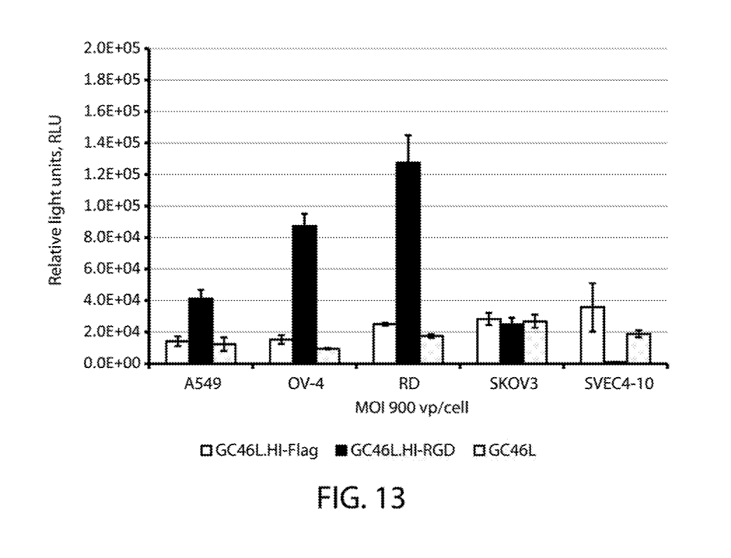

In these experiments, plasmids were transformed into A549 (lung), OV-4 (ovary), RD, SKOV3 (ovary), RD (muscle) and SVEC4-10 cell (endothelial) lines, Expression was measured by Relative Light Units (RLU) indicating reporter luciferase gene expression (FIG. 10-13). AD5L/G-RGD showed better integration than AD5L/G, with a maximum of 7.0×10⁶ relative light units detected (FIG. 10). GC46L.HI-RGD showed better incorporation than either GC46.HI-Flag or untagged GC46L in most cell lines, although in SKOV3 all constructs showed equal integration, and in SVEC4-10, very little GC46L.HI-RGD was detected, but GC46L.HI-Flag expressed well (FIG. 11). In general, GC46L.HI-RGD upper expressed higher than GC46L.HI-RGD, except in the OV-4 cell line Where they were about equal (FIG. 12). GC46L.HI-RGD integrated more readily relative to GC46L.HI-FLAG or GC4GL (FIG. 13).

### Example 18

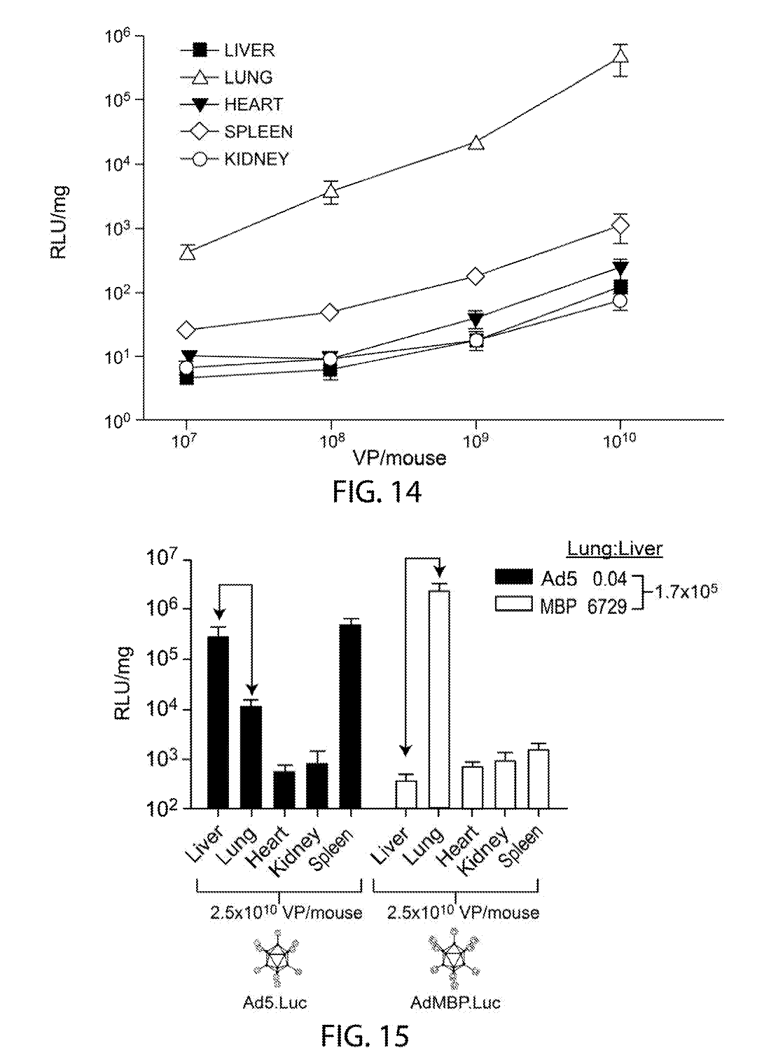

This example illustrates lung targeting of Ad.MBP in vivo. C57BL/6J mice were transformed with Ad.MBPLuc, and expression of MBP Luc was measured with luciferase assay. FIG. 14 illustrates the biodistribution of the luciferase construct in various tissues in different concentrations. Targeting index, lung/liver is 94 at 10⁷ vp/mouse, 645 at 10⁸ vp/mouse, 1218 at 10⁹ vp/mouse, and 3953 at 10¹⁰ vp/mouse. FIG. 15 compares the biodistribution of expression of Ad.5 Luc to AdMBP.Luc in C57BL/6J mice. Ad5.Luc expresses highly in the liver, which can cause liver toxicity. In contrast, AdMBP.Luc is expressed highly in the lungs and very lowly in the liver, allowing for high expression of constructs with low liver toxicity.

### Example 19

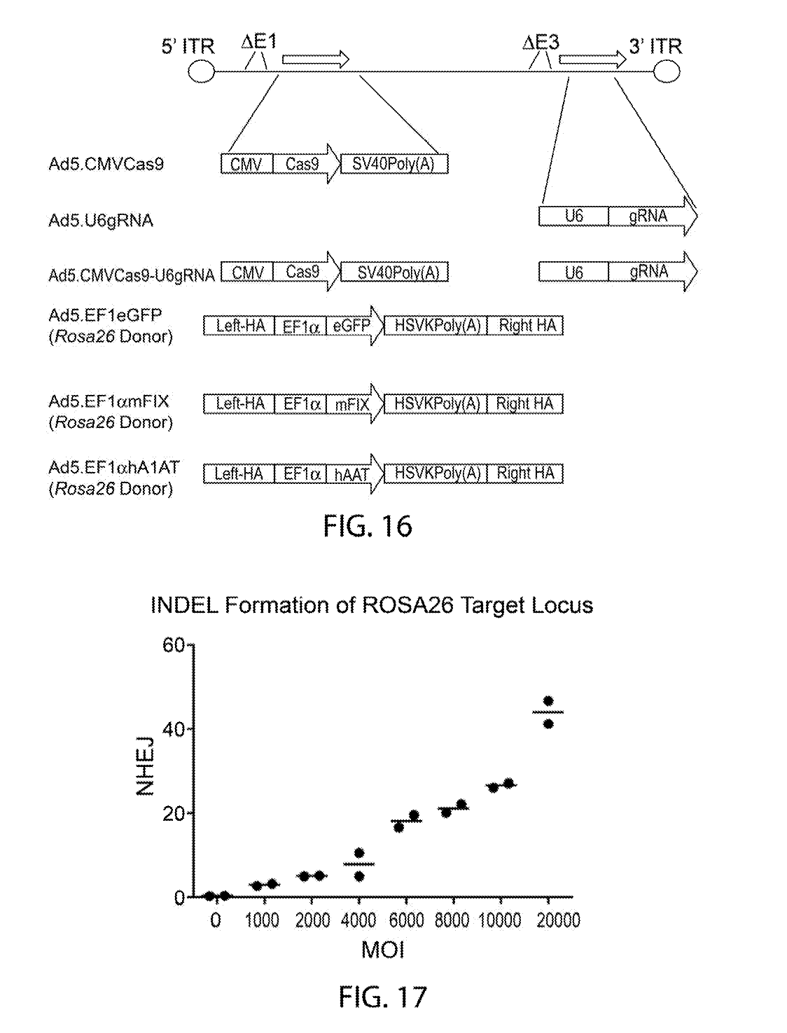

This example illustrates Cas/CRISPR targeting constructs using Adenovirus constructs described in FIG. 16. The first vector set consists of CRISPR components. Control vector Ad.CMVCas9 expresses Cas9 from the E1 deleted region. Control vector Ad.U6gRNA expresses a ROSA26 specific gRNA from the E3 deleted region. The experimental vector Ad.CMVCas-9-UgRNA expresses Cas9 from the E1 deleted region and a ROSA26 specific gRNA from the E3 deleted region. The second vector set contains donor cassettes for integration composed of transgenes (EGFP reporter or secretable A1AT) driven by the constitutive EF1α promoter/intron flanked by up- and downstream sequences (˜0.8 Kb) surrounding the ROSA26 gRNA restriction site. These vectors are: Ad5.EF1eGFP, Ad5-αmFIX, and Ad5.EF1αhA1AT (FIG. 16).

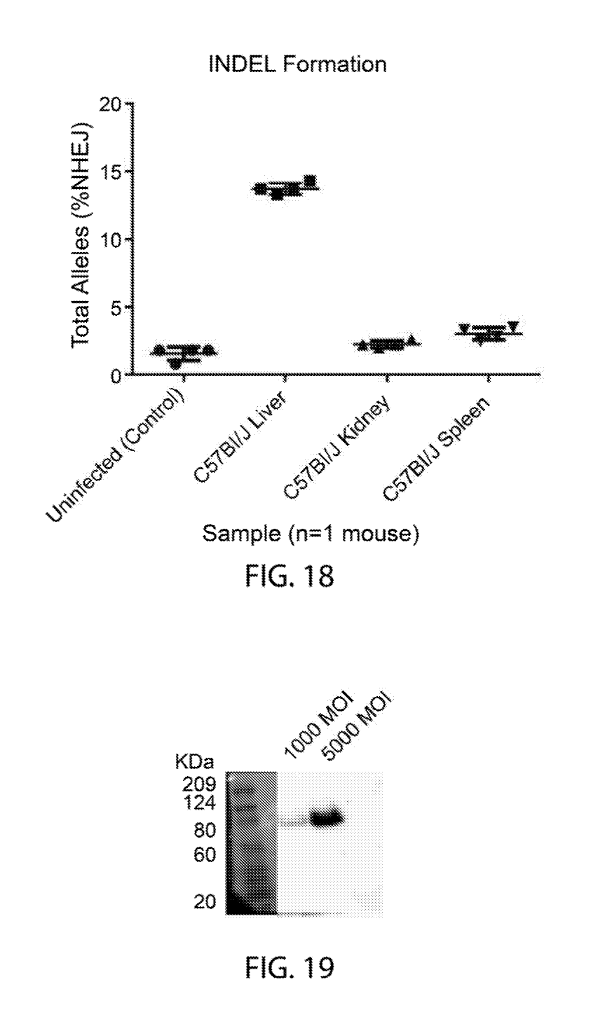

To test Rosa targeting, Ad5.CMV-Cas9-gRNA transduced into BNL-1NG cells. Targeted Illumina deep sequencing of the ROSA26 from genomic DNA of BNL-1NG murine liver cells shows insertions and deletions (indel) formation resulting from NHEJ DNA repair of DSBs increases with increasing multiplicity of infection with Ad5.CMV-Cas9.U6-gRNA (FIG. 17). Furthermore, a C57Bl/J mouse injected with Ad5Cas9gRNA showed 15% INDEL formation in liver, and near control levels the kidney and spleen (FIG. 18).

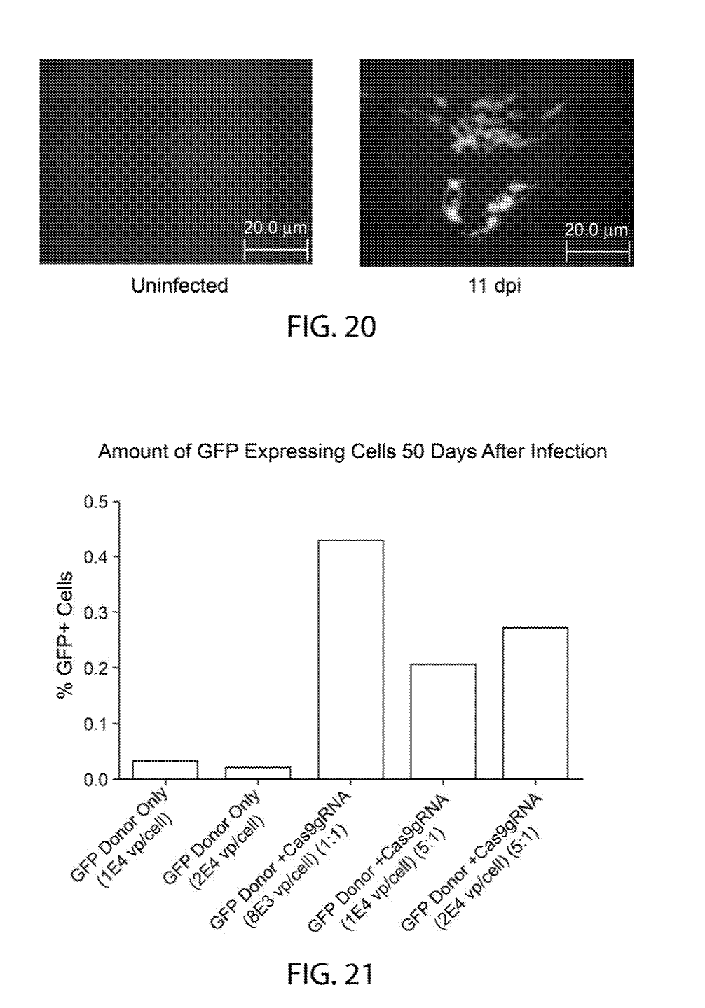

Ad5.EFαmFIX was transformed into A549 cells at 1000 and 5000 MOI. A Western blot against FIX antibody was performed, and increased FIX expression was observed with increased MOI (FIG. 19). A549 cells were infected with Ad.5EF1αGFP at 10,000 vp/cell and expression was observed at 11 dpi (FIG. 20). Further cell transformations using the constructs indicated in FIG. 21 also illustrated stability of Cas9 constructs 50 days after infection. These experiments illustrate that vectors of the present teachings can be used to stably transform genes into mammalian cells.

### Example 20

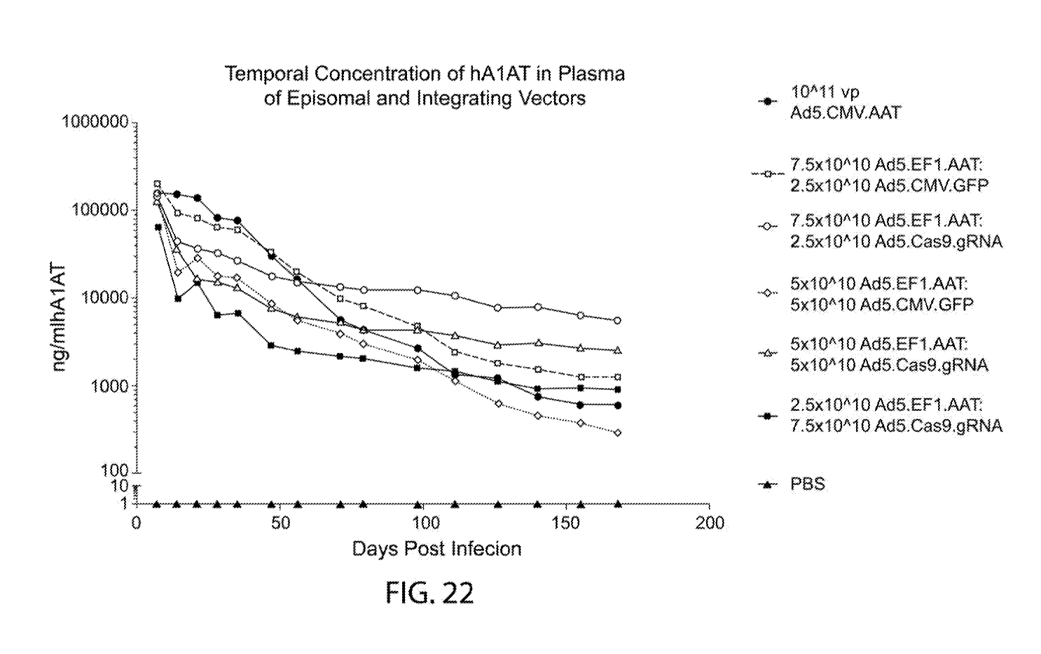

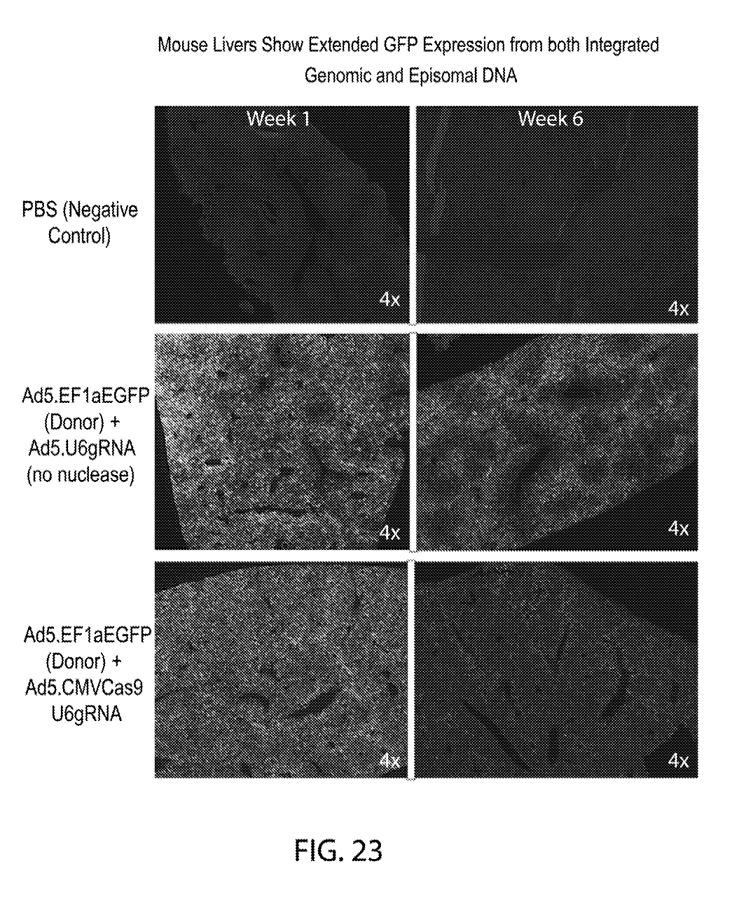

This example illustrates the stability of expression using constructs of the present teachings. As shown in FIG. 22, non-integrative mouse groups were injected with either PBS (filled triangle), 7.5E10 VP of hA1AT donor vector (Ad5.EF1α-hA1AT) and 2.5E10 sham vector (Ad5.CMV-EGFP, open square, broken line) or 5E10 VP of each (open diamond, dotted line). Integrative mouse groups were injected with 7.5E10 VP of hA1AT donor vector (Ad5.EF1α-hA1AT) and 2.5E10 CRISPR-containing vector (Ad.5.CMV-Cas.U6-gRNA, open circle), 5E10 VP of each (open triangle), or 2.5E10 VP of hA1AT donor vector (Ad5.EF1α-hA1AT) and 7.5E10 CRISPR-containing vector (Ad5.CMV-Cas.U6-gRNA, filled square). Plasma levels of hA1AT were determined weekly or bi-weekly via ELISA demonstrating that hA1AT levels were more stable over time in mice receiving a CRISPR/Cas9 integration system compared to an equivalent episomal based expression system.

This effect without a targeting moiety is further illustrated in FIG. 23, which illustrates that Ad constructs can be expressed at week 1 (left column) and week 6 (right column) in mouse liver cells (amplified 4×). These experiments further illustrate the stability of transformations performed using the present teachings.

### REFERENCES

1. Krasnykh, V. N., et al., J. Virol. 1996; 70:6839-6846.
2. Krasnykh, V., et al, J. Virol. 2001; 75:4176-4183.
3. Noureddini, S. C., et al., Virus Res. 2006; 116:185-195.
4. Alberti, M. O., et al., Gene Ther. 2013; 20:733-741.
5. Alberti, M. O., et al., PLoS One 2012; 7:e37812.
6. Zanolini, D., et al., Haematologica 2014; 100: 881-92.
7. Buggio, M., et al., The Journal of Gene Medicine 2016; 18: 38-44.
8. Douglas, J. T et al., 1999; 17: 470-475.
9. Mittereder, N., et al., J. Virol. 1996; 70: 7498-7509.
10. Kaliberov, S. A., et al., Virology, 2013, 447: 312-325.
11. Ehrhardt, A., et al., Molecular Therapy 2007; 15:146-156.
12. Yant, S. R., et al., Nature Biotechnology 2002; 20: 999-1005.
13. Brunetti-Pierri, N. and Ng, P., Human Molecular Genetics 2011; 20: R7-R13.
14. Isman, O., et at, Human Gene Therapy 2008; 19: 1000-1008.
15. Wang, D., et al., Human Gene Therapy 2015; 26: 432-442.
16. Xue, W., et al., Nature 2014; 514: pp. 380-384.
17. Ding, Q., et al., Circulation Research 2014; 115: 488-492.
18. Cheng, R., et al., FEBS Letters 2014; 588: 3954-3958.
19. Tabebordbar, M., et al., Science 2016; 351: 407-411.
20. Yang, Y., et al., Nature Biotechnology 2016:334-338.
21. Nelson, C. E., et al., Science 201.6; 351; 403-407.
22. Yin, H., et al., Nature Biotechnology 2016; 34:328-333.
23. Barzel, A., et al., Nature 2015; 517: 360-364.

All publications cited herein are incorporated by reference, each in its entirety. Applicant reserves the right to challenge any conclusions presented by the authors of any reference.

What is claimed is:

1. An adenovirus vector comprising: a pulmonary targeting coding sequence, a Cas9 coding sequence, and a guideRNA coding sequence.

2. An adenovirus vector in accordance with claim 1, wherein the pulmonary targeting coding sequence is an MBP targeting ligand coding sequence.

3. An adenovirus vector in accordance with claim 1, wherein the MBP targeting ligand coding sequence is a fiber-fibritin-MBP targeting ligand coding sequence.

4. An adenovirus vector in accordance with claim 1, wherein the pulmonary targeting sequence comprises a vascular-specific promoter and integrin targeting peptides incorporated into a viral knob.

5. (canceled)

6. An adenovirus vector in accordance with claim 1, wherein the adenovirus vector is a gorilla adenoviral vector.

7. (canceled)

8. An adenovirus vector in accordance with claim 1, wherein the guideRNA encodes one or more sequences targeted to a ROSA26 locus.

9. A two vector system comprising a first vector in accordance with claim 1 and a second vector comprising left and right homology arms that are homologous to the guideRNA.

10-11. (canceled)

12. A two vector system in accordance with claim 9, wherein the second vector further comprises an EF1α promoter that includes an intron.

13-15. (canceled)

16. A two vector system in accordance with claim 9, wherein the second vector further comprises a Factor IX gene.

17. (canceled)

18. A two vector system in accordance with claim 9, wherein the guideRNA and the homology arms comprise sequences targeted to a ROSA26 locus.

19. A two vector system in accordance with claim 18, wherein the second vector further comprises a Factor IX gene disrupting the sequence of the ROSA26 encoding homology arms.

20. A two vector system in accordance with claim 9, wherein the pulmonary targeting coding sequence is a fiber-fibritin-MBP targeting ligand coding sequence, the Cas9 coding sequence is a codon-optimized coding sequence, the adenovirus vector is a gorilla adenovirus vector, the guideRNA is under the control of the Pol III promoter, and wherein the guideRNA and homology arms encode one or more sequences targeted to a ROSA26 locus and the second vector further comprises a Factor IX gene disrupting the sequence of the ROSA26 encoding homology arms.

21-39. (canceled)

40. An adenovirus vector comprising:
a chimeric AD5-T4 phage fibritin shaft;
a trimerization domain displaying a pulmonary targeting coding sequence; and
a ROBO4 enhancer/promoter operatively linked to a transgene of interest.

41. An adenovirus vector in accordance with claim 40, wherein the transgene of interest is selected from the group consisting of a CRISPR gene, a Cas9 gene, a guideRNA, a hemophilia factor, and a combination thereof.

42-45. (canceled)

46. An adenovirus vector in accordance with claim 41, wherein the transgene of interest comprises a guideRNA which encodes one or more sequences targeted to a ROSA26 locus.

47. (canceled)

48. An adenovirus vector in accordance with claim 40, wherein the transgene of interest encodes a hemophilia factor selected from the group consisting of factor VIII and factor IX.

49. (canceled)

50. A two vector system comprising a first vector in accordance with claim 40 and a second vector comprising left and right homology arms that are homologous to a guideRNA.

51-56. (canceled)

57. A two vector system in accordance with claim 50, wherein the second vector further comprises a Factor IX gene.

58. A two vector system in accordance with claim 50, wherein the pulmonary targeting coding sequence is an MBP targeting ligand coding sequence.

59. A two vector system in accordance with claim 50, wherein the guideRNA and the homology arms comprise sequences targeted to a ROSA26 locus.

60. A two vector system in accordance with claim 59, wherein the second vector further comprises a Factor IX gene disrupting the sequence of the ROSA26 encoding homology arms.

61. A two vector system in accordance with claim 59, wherein:
the pulmonary targeting coding sequence is a fiber-fibritin-MBP targeting ligand coding sequence;
the adenovirus vector is a gorilla adenoviral vector;
the guideRNA is under the control of the Pol III promoter;
the guideRNA and homology arms encode one or more sequences targeted to a ROSA26 locus; and
the second vector further comprises a Factor IX gene disrupting the sequence of the ROSA26 encoding homology arms.

62. A method of gene therapy comprising administering to a subject an adenovirus comprising a two vector system in accordance with claim 50.

63-64. (canceled)
